(19) United States
(12) Patent Application Publication
(10) Pub. No.: US 2006/0067957 A1
(43) Pub. Date: Mar. 30, 2006

(54) Self-heating cosmetic products

(76) Inventors: Donna Hui-Ing Hwang, Leonia, NJ (US); Domnica Cernasov, Ringwood, NJ (US); Ralph Macchio, Sparta, NJ (US)

Correspondence Address: PENDORF & CUTLIFF, 5111 MEMORIAL HIGHWAY, TAMPA, FL, 33634-7356, US

(21) Appl. No.: 10/542,536

(22) PCT Filed: Jan. 16, 2004
(86) PCT No.: PCT/EP04/00317

§ 371 Date: Jul. 18, 2005

(30) Foreign Application Priority Data
Jan. 16, 2003 (DE) 103 02 096.9

(51) Int. Cl. A61K 8/37; A61K 8/25

(52) U.S. Cl. 424/401

(57) ABSTRACT

The invention refers to cosmetic products having a self-warming effect. The composition is substantially water-free and contains 0.1 to 40% by weight of a hydrophilic aluminum-rich zeolite with a pore size in the range of 0.3 to 0.5 nm, 1 to 99% by weight of a disperse oil phase, 0.1 to 50% by weight of a non-ionic surface-active agent, 0.1 to 40% by weight of an emollient, 0.1 to 20% by weight of a thickener, and up to 100% by weight cosmetic carrier substances, auxiliary substances, active substances and mixtures thereof, with the provisio that no polyvalent alcohols and polar solvents are contained. The products have an enhanced water absorption, cleansing effect and a silk-like feeling.

### BACKGROUND OF THE INVENTION

1. Field of the Invention

The invention refers to cosmetic products having a self- warming effect.

2. Related Art of the Invention

U.S. Pat. No 6,309,655 discloses a cosmetic composition containing an self-warming component and consisting of a granulated material which indicates its own disintegration, the said granulated material containing a water-insoluble polymer, a colourant and a water-free carrier which is mixable with water. The water-insoluble polymer is e.g. a polyethylene, polystyrene, polyvinyl chloride, polyacrylic resin, etc. and the carrier which is mixable with water is a polyvalent alcohol, a sugar alcohol and ethylene adducts of different sugars and/or a zeolite.

WO 00/38621 describes an self-warming hair conditioner consisting of a microporous material the pore sizes of which are greater than water molecules, such as e.g. an alkali silicate, bentonite, diatomaceous earth, a carrier molecule the critical diameter of which is greater than the microporous material, such as e.g. hydrophilic glycols, polyethylene glycols, glycerine, etc., and a conditioning molecule the diameter of which is greater than the microporous material, such as e.g. quaternary ammonium compounds, cationic polymers and long-chain hydrocarbons.

### SUMMARY OF THE INVENTION

The object of the invention is to provide a cosmetic self-warming product having an improved water absorption capacity and thus an stronger warming effect.

Another object of the invention is to provide a product which leaves a particularly soft, silk-like feeling on the skin.

Another object of the invention is to provide a cleansing product with self-warming effect.

### DETAILED DESCRIPTION OF THE INVENTION

According to the invention a cosmetic self-warming product is provided which is substantially water-free and contains

to 40% by weight of a hydrophilic aluminum-rich zeolite with a pore size in the range of 0.3 to 0.5 nm, to 99% by weight of a disperse oil phase, to 50% by weight of a non-ionic surface-active agent, to 40% by weight of an emollient, to 20% by weight of a thickener, and ad 100% by weight cosmetic carrier substances, auxiliary substances, active substances and mixtures thereof, with the proviso that no polyvalent alcohols and polar solvents are contained.

In contrast to known cosmetic products the presence of zeolites and the thermal effect which occurs when brought into contact with water cause the pores of the skin to open so that the oil used as conditioning/cleansing agent can act more effectively. In contrast to known cosmetic self-warming products, which as a rule contain polyvalent alcohols and zeolites, the absence of polyvalent alcohols considerably reduces the water absorption potential. The zeolite contained in the product can thus develop its full thermal effect at the time when this is desired and not before. If the water absorption potential is high due to other components the addition of a few tenths per cent of water can result in that the zeolite warms up thus partly reducing the overall thermal potential thereof.

The product according to the invention has a particularly intense warming effect and leaves a special, silk-like feeling on the skin once the product has been applied.

The more zeolite develops its thermal effect due to the exothermal reaction with water the better is the pore opening effect and the more intense is the conditioning/cleansing effect. In addition, a certain water-repellent effect is achieved due to the combination of a high oil content in the product according to the invention with the zeolite and the absence of polyvalent alcohols, which water-repellent effect contributes to the product being extremely stable and maintaining all its desired properties when stored.

Advantageously, the product according to the invention can additionally contain an oil absorbent in amounts in the range of 0.1 to 20% by weight relative to the total weight of the cleanser in order to reduce stickiness or a fatty feeling. Therefore zinc stearate, kaolin, kaolin according to WO 96/17588, Aluminium Starch Octenyl Succinate and similar products can be contained.

Kaolin according to WO 96/17588 is a kaolin which has been modified with spherical TiO₂ or SiO₂ particles the particle size of which is <5 μm, the said spherical particles making up 0.5 to 10% by weight of the kaolin mixture.

Further, it is advantageous to add a slip modifier in amounts in the range of 0.1 to 20% by weight relative to the total weight of the cleanser, such as e.g. Talc, Silica, Trimethyl Siloxysilicate, etc. In this way, the product is given very good tactile aesthetics and a very good feeling when applied onto the skin.

When brought into contact with water the best effect is achieved if approximately 50% by weight of water are used relative to the weight of the product according to the invention used. However, the thermal effect is already noticeable if just 0.5% by weight of water are used and is still noticeable if approximately 300% by weight of water are added, in both cases relative to the weight of the product according to the invention. The thermal effect caused by the zeolite results in an increase in temperature by 4 to 8 K, compared to the surface temperature of the skin to be treated.

The zeolite used is present in a completely dry form. Preferably, a hydrophilic synthetic zeolite which is rich in aluminium is used, such as Zeolite A, NaX, NaY or a natural zeolite having similar properties. A preferred aluminum-rich zeolite has a ratio Si/Al in the range of 2-5:1. The medium pore size of the zeolite is generally in the range of 0.3 to 0.5 nm in order to enable water molecules to enter the pores. The zeolite content of the product is preferably in the range of 15 to 25% by weight or 12-28% by weight.

In the context of the present invention “substantially water-free” means that no water is added to the product and water is contained only in the form of constitutional water which in some cases cannot be avoided and is brought in as part of the ingredients in very small amounts.

In the present cosmetic composition the disperse oil phase can be formed by non-polar, semi-polar and polar oils as well as esters and ethers. These include mineral oils, triglycerides, silicone oils, Hydrogenated Polyisobutene, Polyisoprene, Squalane, Tridecyl Trimellitate, Trimethylpropane Triisostearate, Isodecyl Citrate, Neopentylglycol Diheptanoate, PPG-15 Stearyl Ether, as well as vegetable oils, such as Calendula Oil, Jojoba Oil, Avocado Oil, Macadamia Nut Oil, Castor Oil, Wheat Germ Oil, Grapeseed Oil, Kukui Nut Oil, Thistle Oil, Evening Primrose Oil, or a mixture of several thereof. A preferred dispersed oil which lowers the surface tension of the zeolite such as Caprylic/Capric Triglyceride. All oils used are anhydrous oils.

Further, the formulation preferably has a high oil content of 20 to 70% by weight, particularly an oil content of 25 to 50% by weight. The anhydrous dispersed oil functions also as a dispersant to enhance the performance and esthetic efficiency of zeolite in the formulation without using shear to achieve a good dispersion. Zeolite in a powder form is not soluble in most solvents and thus achieving a good suspension is always a problem. Normally it requires a very high energy input by shear to achieve such a good suspension. This is avoided by the special dispersed oil.

The non-ionic surface-active agent is an ester, ether, alkanol amide or amine oxide, preferably a sorbitan ester, Trialkyl Phosphate, Ethoxylated Alcohol, Ethoxylated Castor Oil, Ethoxylated Polysiloxane, Alkyl Glycoside, a POE/PPG ester, Palmitamide-DEA, Dihydroxyethyl Stearamine Oxide, Methoxylated Glyceride, Lecithin, etc.

The non-ionic surface-active agent, such as e.g. Tween 60 or Lecithin, enables the cleansing composition to be washed off the skin easier.

The preferred amount of the surface-active agent is in the range of 3 to 15% by weight.

Further, the composition contains 0.1 to 40% by weight, preferably 10 to 25% by weight, of an emollient. Such emollients are e.g. esters of fatty acids having 12 to 18 carbon atoms in the alkyl part of the acid. It is preferred that the alcohol rest be a linear-chain, branched or cyclic hydrocarbon having 10 to 40 carbon atoms, a glyceryl ester or a low-molecular α-olefin. Examples are Glyceryl Monoricinoleate, Glyceryl Monostearate, Isopropyl Isostearate, Isobutyl Palmitate, Isopropyl Laurate, Isopropyl Myristate, Decyl Oleate, Cetyl Palmitate; or silicone oils, such as Dimethyl Polysiloxane, or Lanolin and Cocoa Butter.

Further, the composition contains 0.1 to 20% by weight, preferably 0.2 to 10% by weight, of a thickener. The thickener can modify and increase the viscosity of the oil and build up the structure of the phase. Polyethylene, 12-Hydroxy Stearic Acid, silicone copolymers, clay, castor oil derivatives and waxes can be used as thickener. Examples are polyethylene having a molecular weight in the range of 300 to 600; and waxes, such as Carnauba Wax, Candelilla Wax, Beeswax, Wool Wax, Paraffin, Ceresin, Ozokerite, Silicone, microcrystalline wax or polyethylene glycol ester waxes. Preferred thickeners are wax and castor oil derivatives.

Further, the product according to the invention contains cosmetic auxiliary substances and carrier substances as they are commonly used in such preparations, e.g. preservatives, colourants, fragrances, gel-forming agents, polymers, copolymers, emulsifiers, stabilizers.

Additional cosmetic active agents which can be used include e.g. inorganic and organic sunscreens, scavengers, moisturizing substances, vitamins, enzymes, vegetable active agents, antioxidants, anti-inflammatory natural active agents.

The cosmetic product according to the invention can e.g. be used in a cleansing milk, in face masks, hair masks, lotions and other cleansing compositions. These products are manufactured in a way which is known to those skilled in the art.

The invention will hereinafter be explained in detail by way of examples. All quantities are given in % by weight if not indicated otherwise.

### EXAMPLE 1

### Warming Message Balm Formula

| Phase | Ingredients | wt. % |
|---|---|---|
| A | caprylic/capric triglyceride | 35-40 |
| | C18-36 acid glycolester | 2-5 |
| | synthetic wax | 2-5 |
| B | isopropyl palmitate | 20-25 |
| | Zeolite¹ | 18-22 |
| | Pigment | qs. |
| C | Preservatives | qs. |
| | PPG-15 stearyl ether | 8-12 |
| D | tocopheryl acetate | qs. |
| | Fragrance | qs. |
| | Total | 100.0 |

¹Potassium form of zeolite A (Molsiv ® GMP3A of UOP LLC, Des Plaines, IL, USA).

Add ingredients of Phase A into a clean, dry, stainless steel kettle equipped with a mixer. With slow mixing, begin to heat batch to 80° C. and maintain temperature until batch is uniform with no un-dissolved raw materials present. Separately premix the ingredients of Phase B until uniform. Add Phase B to Phase A with moderate mixing speed. Mix 15 minutes or longer if necessary until the batch is uniform. Continue mixing, add the ingredients of Phase C. to Phase A+B and maintain batch temperature at 70-75° C. until uniform. Discontinue heating and begin to cool batch to 50° C. When batch temperature reaches 50° C., add fragrance to the main batch kettle. Mix well until batch is uniform and homogeneous.

### Example 2

### Warming Butter Cream Formula

| Phase | Ingredients | wt. % |
|---|---|---|
| A | caprylic/capric triglyceride | 30-35 |
| | Trihydroxystearin | 0.5-1 |
| B | microcrystalline wax | 2-5 |
| | C18-36 acid glycol ester | 0.5-1 |
| | Tribehenin | 2-4 |
| | BHT (dibutylhydroxytoluen) | qs. |
| | Preservatives | qs. |
| C | isopropyl palmitate | 15-20 |
| | corn starch | 10-15 |
| | Zeolite | 20-25 |
| | Pigment | qs. |
| D | tocopheryl acetate | qs. |
| | Fragrance | qs. |
| | Total | 100.0 |

Add ingredients of Phase A into a clean, dry, stainless steel kettle equipped with a mixer. With slow mixing, begin to heat batch to 80° C. and maintain temperature until batch is uniform with no un-dissolved raw materials present. Continue mixing, add the ingredients of Phase B to Phase A and maintain batch temperature at 80° C. until uniform. In a separate stainless steel vessel, premix the ingredients of Phase C until uniform. Add Phase C to Phase A+B with moderate mixing speed. Mix 15 minutes or longer if necessary and maintain batch temperature at 70-75° C. until uniform. Discontinue heating and begin to cool batch to 50° C. When batch temperature reaches 50° C., add fragrance to the main batch kettle. Mix well until batch is uniform and homogeneous.

### Example 3

### Warming Foot Scrub Formula

| Phase | Ingredients | wt. % |
|---|---|---|
| A | caprylic/capric triglyceride | 28-33 |
| | trihydroxystearin | 3-6 |
| B | polyethylene | 1-3 |
| | synthetic wax | 1-5 |
| | C18-36 acid glycol ester | 1-5 |
| | BHT | qs. |
| C | caprylic/capric triglyceride | 8-12 |
| | isopropyl palmitate | 8-15 |
| | quartz and pumice | 6-10 |
| | zeolite | 15-22 |
| D | polysorbate 60 | 6-10 |
| E | preservatives | qs. |
| | fragrance | qs. |
| | Total | 100.0 |

Add ingredients of Phase A into a clean, dry, stainless steel kettle equipped with a mixer. With slow mixing, begin to heat batch to 80° C. and maintain temperature until batch is uniform with no un-dissolved raw materials present. Continue mixing, add the ingredients of Phase B to Phase A and maintain batch temperature at 80° C. until uniform. In a separate stainless steel vessel, premix the ingredients of Phase C until uniform. Add Phase C to Phase A+B with moderate mixing speed. Mix 15 minutes or longer if necessary until the batch is uniform. Continue mixing, add the ingredients of Phase D to Phase A+B+C and maintain batch temperature at 80° C. until uniform. Discontinue heating and begin to cool batch to 50° C. When batch temperature reaches 50° C., add fragrance and preservative into batch and continue to mix until batch is uniform.

### Example 4

### Warming Cleanser Formula

| Phase | Ingredients | wt. % |
|---|---|---|
| A | caprylic/capric triglyceride | 35-40 |
| | trihydroxystearin | 3-6 |
| B | polyethylene | 1-3 |
| | BHT | qs. |
| C | caprylic/capric triglyceride | 9-16 |
| | triethyl hexanoin | 4-6 |
| | polyglyceryl-3-laurate | 4-6 |
| | zeolite¹ | 20-25 |
| | kaolin | 1-3 |
| D | polysorbate 60 | 4-8 |
| | lecithin | 1-3 |
| E | preservatives | qs. |
| | fragrance | qs. |
| | Total | 100.0 |

¹Sodium form of zeolite A (Molsiv ® GMP 4A of UOP, Mt. Laurel, NJ, USA.

Add ingredients of Phase A into a clean, dry, stainless steel kettle equipped with a mixer. With slow mixing, begin to heat batch to 80° C. and maintain temperature until batch is uniform with no un-dissolved raw materials present. Continue mixing, add the ingredients of Phase B to Phase A and maintain batch temperature at 80° C. until uniform.

In a separate stainless steel vessel, premix the ingredients of Phase C until uniform. Add Phase C to Phase A+B with moderate mixing speed. Mix 15 minutes or longer if necessary until the batch is uniform. Continue mixing, add the ingredients of Phase D to Phase A+B+C and maintain batch temperature at 80° C. until uniform. Discontinue heating and begin to cool batch to 50° C. When batch temperature reaches 50° C., add fragrance and preservative into batch and continue to mix until batch is uniform.

1-9. (canceled)

10. A cosmetic self-warming product for skin cleansing, comprising a composition which is substantially water-free and contains:
0.1 to 40% by weight of a hydrophilic aluminum-rich zeolite with a pore size in the range of 0.3 to 0.5 nm, 1 to 99% by weight of a disperse oil phase, selected from the group consisting of triglycerides, silicone oils and mixtures thereof,
0.1 to 50% by weight of a non-ionic surface-active agent,
0.1 to 40% by weight of an emollient,
0.1 to 20% by weight of a thickener, selected from the group consisting of polyethylene, 12-hydroxy stearic acid, clay, castor oil derivatives and waxes, and
ad 100% by weight cosmetic carrier substances, auxiliary substances, active substances and mixtures thereof,
with the proviso that no polyvalent alcohols and polar solvents are contained.

11. The product according to claim 10 wherein the product contains an oil absorbent selected from the group consisting of zinc stearate, kaolin and mixtures thereof.

12. The product according to claim 10 wherein the product contains a zeolite with a ratio Si/Al in the range of 2-5:1.

13. The product according to claim 10 wherein the zeolite content is in the range of 15 to 25% by weight.

14. The product according to claim 10 wherein the dispersed oil content is in the range of 20 to 70% by weight.

15. The product according to claim 10 wherein the dispersed oil is anhydrous Caprylic/Capric Triglyceride.

16. The product according to claim 10 wherein the thickener is 0.2 to 10% by weight wax or castor oil derivative or a mixture thereof.

17. A method for cleaning skin, comprising applying to skin a composition comprising:
0.1 to 40% by weight of a hydrophilic aluminum-rich zeolite with a pore size in the range of 0.3 to 0.5 nm,
1 to 99% by weight of a disperse oil phase selected from the group consisting of triglycerides, silicone oils and mixtures thereof,
0.1 to 50% by weight of a non-ionic surface-active agent,
0.1 to 40% by weight of an emollient,
0.1 to 20% by weight of a thickener selected from the group consisting of polyethylene, 12-hydroxy stearic acid, clay, castor oil derivatives and waxes, and
ad 100% by weight cosmetic carrier substances, auxiliary substances, active substances and mixtures thereof,
with the proviso that no polyvalent alcohols and polar solvents are contained,
in combination with 10 to 100% by weight of water relative to the weight of the product for thorough cleansing of the skin;
wherein said composition is exothermic on contact with water and is formulated to cause a raise in temperature of the skin by 4 to 8 K during cleansing compared to the starting surface temperature of the skin.

18. The method according to claim 17, wherein for cleansing said composition is mixed with a further 40 to 60% by weight water based on the weight of the starting composition.

19. The method according to claim 17, wherein said composition is in the form of a hand cream, face mask, foot scrub or lotion.
